(19) United States
(12) Patent Application Publication
(10) Pub. No.: US 2026/0207546 A1
(43) Pub. Date: Jul. 23, 2026

(54) METHOD OF TREATING AND/OR PREVENTING FAT-RELATED METABOLIC DISEASE USING GENISTEIN OR GENISTEIN-7-O-PHOSPHATE

(71) Applicants: Tunghai University, Taichung City (TW); National Taiwan University, Taipei City (TW)

(72) Inventors: Yu-Hsuan Lin, Taoyuan City (TW); Yung-Kai Lin, Taichung City (TW); Tzu-Tang Wei, Taipei City (TW); Nan-Wei Su, Taipei City (TW); Vipul Wayal, Dist-Pune (IN); Chang-Chi Hsieh, Taichung City (TW)

(21) Appl. No.: 19/343,134

(22) Filed: Sep. 29, 2025

(30) Foreign Application Priority Data
Jan. 20, 2025 (TW) 114102385

(51) Int. Cl. A61K 31/352; A61K 31/665; A61P 1/16; A61P 3/04; A61P 3/06

(52) U.S. Cl. CPC A61K 31/352; A61K 31/665; A61P 1/16; A61P 3/04; A61P 3/06

(57) ABSTRACT

A method of treating and/or improving a metabolic disease using genistein or genistein-7-O-phosphate includes administering an effective amount of genistein or genistein-7-O-phosphate to an individual to inhibit the accumulation of fat in the individual's body, e.g., in/around the individual's internal organs, thereby producing the effect of treating and/or preventing a disease related to abnormality in fat metabolism.

### CROSS-REFERENCE TO RELATED APPLICATIONS

This application claims the benefit of Taiwan Patent Application No. 11402385, which was filed on Jan. 20, 2025.

### FIELD OF THE INVENTION

The present invention relates to a method of treating or preventing a metabolic disease. More particularly, the invention relates to a method of treating and/or preventing a fat-related metabolic disease using genistein or genistein-7-O-phosphate.

### DESCRIPTION OF THE RELATED ART

The World Health Organization has identified obesity as a chronic disease. Moreover, obesity increases the chance of the development of such diseases as diabetes, metabolic syndrome, hyperlipidemia, hypertension, cardiovascular diseases, and joint diseases. The main cause of obesity is that a far higher calorie intake than expended leads to the accumulation of excess calories in the form of body fat, and in addition to dietary and physical activity habits, factors that may contribute to obesity include work stress and genes.

People nowadays are paying more and more attention to their health and physique and therefore tend to prevent obesity by controlling their diet and doing more physical exercise. Most people, however, eat out most of the time and have problem doing physical exercise on a regular basis; as a result, the accumulation of fat cannot be effectively controlled. Some people use fasting to lose weight rapidly, but this method is disadvantageous in that it lowers the metabolic rate and gives rise to muscle loss and nutritional deficiencies. Weight reduction products to be administered orally or otherwise may also be used to improve obesity, and yet most of the weight reduction products on the market are added with illegal ingredients that may cause undesirable physiological responses, if not irrevocable damage to health.

### SUMMARY OF THE INVENTION

The primary objective of the present invention is to provide a method of treating and/or preventing a fat-related metabolic disease using genistein or genistein-7-O-phosphate. It is desirable that by administering an effective amount of genistein, or genistein-7-O-phosphate, or a composition containing either of the above to an individual can effectively reduce or improve the buildup of fat in the individual's body.

To achieve the foregoing objective, the present invention discloses a method of treating and/or preventing a fat-related metabolic disease using genistein (GEN) or genistein-7-O-phosphate (G7P). Also disclosed is a use of genistein or genistein-7-O-phosphate in preparing a soluble epoxide hydrolase (sEH) inhibitor. When an effective amount of genistein, or genistein-7-O-phosphate, or a composition containing either of the above is administered to an individual, the genistein that enters the individual's body can bind to, and thereby inhibit the activity of, the soluble epoxide hydrolase in the body, producing the effect of improving a metabolism-related disease.

The effective dose of genistein-7-O-phosphate in an individual is significantly lower than that of genistein because genistein-7-O-phosphate can be better absorbed by the digestive tract than genistein and, once entering the small intestine, is metabolically reduced to genistein by the epithelial cells of the small intestine.

The aforesaid metabolism-related disease is related to fat metabolism.

The half-maximal inhibitory concentration of genistein against soluble epoxide hydrolase is 10-12 nM.

Another embodiment of the present invention discloses a use of genistein or genistein-7-O-phosphate in preparing a composition for treating and/or preventing a disease related to abnormality in fat metabolism. That is to say, an effective amount of genistein-7-O-phosphate or a composition containing the same can be administered to an individual in order for the genistein-7-O-phosphate to reduce the fat content of the individual's body or body tissue or prevent continuous accumulation of fat in the individual's body or body tissue, thereby producing the effect of preventing and/or treating a disease related to abnormality in fat metabolism.

The aforesaid disease related to abnormality in fat metabolism is, for example, fatty liver, obesity, hyperlipidemia or a disease related thereto, a high body fat content or a disease related thereto, or inflammation caused by fatty liver.

The aforesaid composition may be a drug, food, or a nutritional supplement.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows the test results of the body weight changes of each group of mice during the test.

FIG. 2 shows the appearances of the liver, spleen, and kidneys of each group of mice.

FIG. 3 shows an analysis result of the weights of the liver, spleen, and kidneys of each group of mice.

FIG. 4 shows the appearances of the abdominal fat, mesenteric fat, and perirenal fat of each group of mice.

FIG. 5 shows an analysis result of the weights of the abdominal fat, mesenteric fat, perirenal fat, and total body fat of each group of mice.

FIG. 6 shows the H&E-stained liver tissue section of each group of mice.

FIG. 7 shows the H&E-stained perirenal fat tissue section of each group of mice.

FIG. 8 shows an analysis result of the serum triglyceride level, total cholesterol level, high-density lipoprotein-cholesterol level, and low-density lipoprotein-cholesterol level of the serum of each group of mice.

FIG. 9 shows an analysis result of the triglyceride level and total cholesterol level in the livers of each group of mice.

FIG. 10 shows an analysis result of the IL-6, TSLP, and TNF-α levels in the livers of each group of mice.

FIG. 11 shows an analysis result of the docking between compound33, which served as a comparative drug, and an sEH protein molecule.

FIG. 12 shows an analysis result of the docking between genistein and an sEH protein molecule.

### DETAILED DESCRIPTION OF THE INVENTION

The present invention discloses a method of treating and/or preventing a fat-related metabolic disease using genistein or genistein-7-O-phosphate. More specifically, genistein has the activity to bond to, and thereby inhibit the activity of, the soluble epoxide hydrolase in a living organism and therefore has the activity to reduce the following in the living organism: the body fat content, visceral fat content, and the size and quantity of liver fat; the triglyceride level, cholesterol level, and low-density lipoprotein-cholesterol level of blood; and the expression of inflammation-related hormones. Meanwhile, genistein-7-O-phosphate can be effectively absorbed by the digestive tract of a living organism and converted into genistein by metabolism once entering the body of the living organism. The method disclosed by the invention for treating and/or preventing a fat-related metabolic disease using genistein or genistein-7-O-phosphate involves administering an effective amount of genistein or genistein-7-O-phosphate to an individual to inhibit the accumulation of fat in the individual's body or in/around the individual's internal organs, thereby producing the effect of treating and/or preventing a disease related to abnormality in fat metabolism.

Genistein-7-O-phosphate has an about 80% higher absorption rate than genistein in the digestive tract and therefore can be used at a lower dose in the body of a living organism than genistein to treat and/or prevent a disease related to abnormality in fat metabolism. The higher absorption rate and higher bioavailability of genistein-7-O-phosphate than those of genistein lend high industrial applicability and high economic value to genistein-7-O-phosphate.

The aforesaid individual is a human, a dog, a cat, a pig, or another animal.

The aforesaid disease related to abnormality in fat metabolism is obesity, fatty liver, hepatitis caused by fatty liver, hyperlipidemia, hypercholesterolemia, or a complication of at least one of the above.

The term “genistein-7-O-phosphate (G7P)” refers to a compound derived from genistein and having the following structural formula (I):

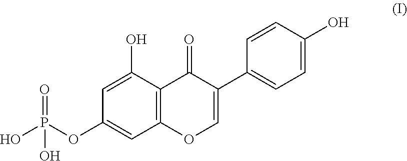 [SMILES: O=c1c(-c2ccc(O)cc2)coc2cc(OP(=O)(O)O)cc(O)c12]

The term “genistein (GEN)” refers to one of the isoflavones that soybean is rich in, and is represented by the following structural formula (II):

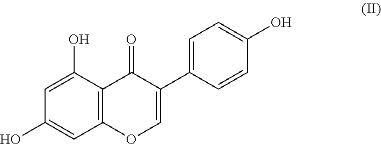 [SMILES: O=c1c(-c2ccc(O)cc2)coc2cc(O)cc(O)c12]

This compound is structurally similar to estrogen and is therefore often referred to as a plant estrogen. Studies show that genistein has such functions as preventing osteoporosis, cardiovascular diseases, breast cancer, and prostate cancer.

The term “soluble epoxide hydrolase (sEH)” refers to an important two-terminal functional enzyme that is widely distributed in such organs as liver, kidney, and brain. Studies show that inhibition of soluble epoxide hydrolase helps maintain the expression of endogenous epoxyeicosatrienoic acids (EETs) and reduce the expression of dihydroxyeicosatrienoic acids (DHETs), thus contributing to the treatment or improvement of cardiovascular diseases, central nervous system diseases, or metabolic diseases.

The term “compound33,” also known as sEH inhibitor-14, refers to a compound having the following structural formula (III):

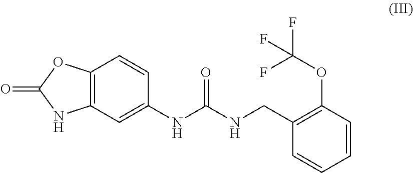 [SMILES: O=C(NCc1ccccc1OC(F)(F)F)Nc1ccc2oc(=O)[nH]c2c1]

The term “disease related to abnormality in fat metabolism” refers to a disease caused by the accumulation of excessive fat in an individual's body or by an excessive intake of fat. Generally, a patient with such a disease has such a symptom as a body fat content and/or blood lipid level higher than the clinical normal value. Diseases related to abnormality in fat metabolism include hyperlipidemia, hypertension, cardiovascular diseases, obesity, metabolic syndrome, diabetes, fatty liver, hepatitis, and so on.

Any numerical value disclosed herein, be it related to “dose”/“dosage” or otherwise, should be construed as including the value itself and a range of errors that is acceptable in the field to which the present invention pertains. More specifically, in the field to which the invention pertains, an acceptable range of errors of a specified value is ±10% of the value. For example, the dose 0.9 nM should be construed as covering the range of 0.9±0.09 nM.

The technical features of the present invention and their effects are described in detail below with reference to some examples in conjunction with the accompanying drawings.

The animal test in the following examples complied with applicable ethical guidelines for animal tests.

The animal test in the following examples was performed on mice by way of example, so the doses/dosages used in the animal test were those intended for mice. That is to say, the doses/dosages are subject to change when the individuals to which the substances in question are administered are changed or when the administration method is changed. The changes in dose/dosage can be calculated by a person of ordinary skill in the art according to the person's general common knowledge.

### Example 1: Animal Test

A plurality of male C57BL/6 mice were divided into 4 groups, and each group of mice was reared under a different condition as stated below:
Group 1: fed with normal feed and normal drinking water
Group 2: fed with high-fat (60% fat) feed and 30% fructose drinking water, in addition to sterile distilled water (10 mL/kg/day) tube-fed on a daily basis
Group 3: fed with high-fat (60% fat) feed and 30% fructose drinking water, in addition to genistein-7-O-phosphate (0.1 mg/kg/day) tube-fed on a daily basis
Group 4: fed with high-fat (60% fat) feed and 30% fructose drinking water, in addition to genistein (1.0 mg/kg/day) tube-fed on a daily basis.

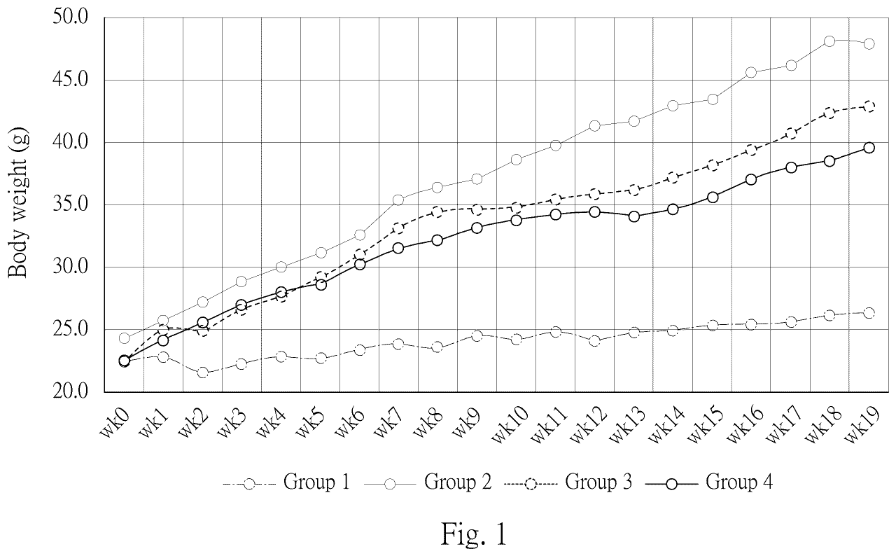

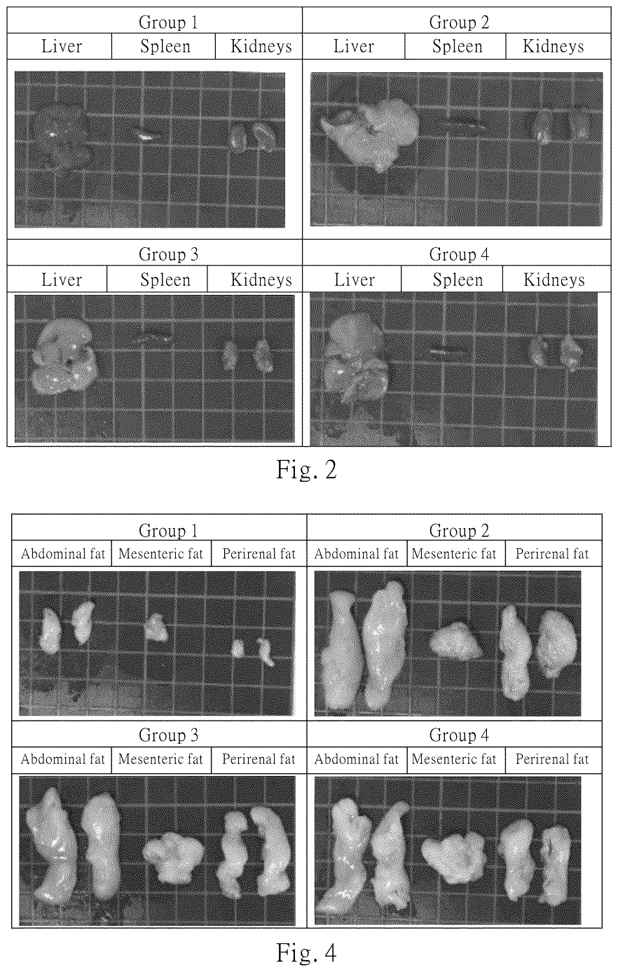

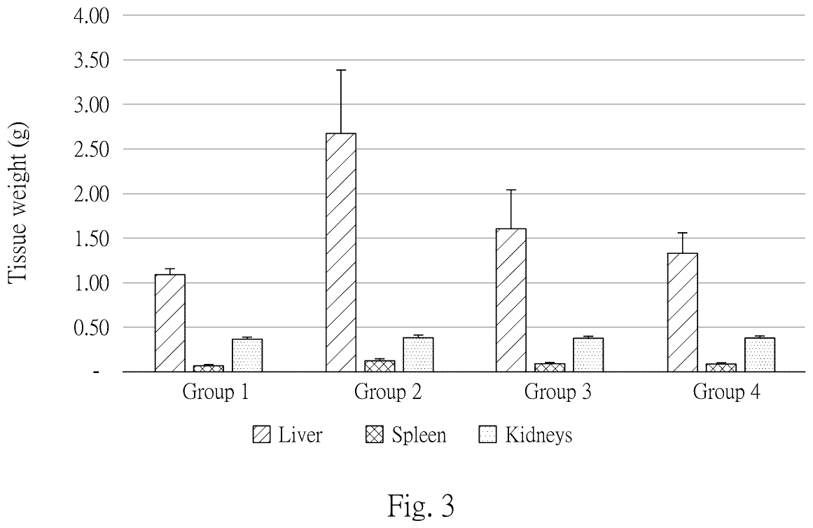

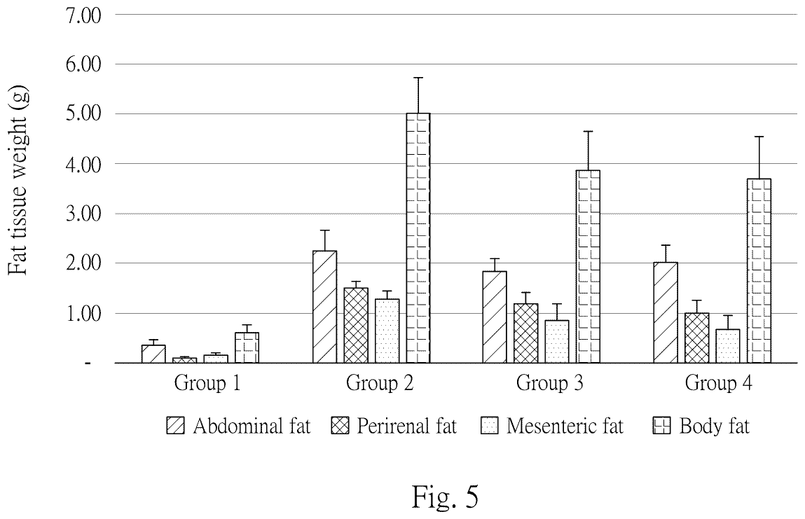

The mice were reared for a total of 19 weeks. During the rearing period, the body weight of each group of mice was measured every week, and the results are shown in FIG. 1. At the end of the rearing period, the mice were sacrificed, and the liver, kidneys, spleen, abdominal fat, mesenteric fat, and perirenal fat of each mouse were taken and photographed, as shown in FIG. 2 and FIG. 4. In addition, the aforesaid organs, fat tissues, and total body fat of each mouse were weighed, and the results are shown in FIG. 3 and FIG. 5. After that, the livers and the fat tissues were embedded in paraffin, sectioned, and stained for subsequent analysis.

It can be known from the results in FIG. 1 to FIG. 5 that, compared with the mice in group 1, the mice in group 2 showed a significant increase not only in body weight, but also in liver, spleen, kidney, and visceral fat weight and/or size, and that the body weight of the mice in group 2 increased with time; therefore, it can be inferred that the high-fat feed and high-sugar drinking water did cause fat to accumulate in/around the organs and in the tissues, resulting in obesity. By contrast, compared with the mice in group 2, the mice in group 3 and group 4 showed a reduction in body weight and a significant reduction in size of the organs and fat tissues.

The foregoing results show that a high-fat and/or high-sugar diet does lead to obesity, and that if genistein-7-O-phosphate or genistein is administered to an individual having such a diet, however, an increase in the individual's body weight and the accumulation of fat in the individual's body can be inhibited. That is to say, each of genistein and genistein-7-O-phosphate is effective in improving a fat-related metabolic disease. Besides, genistein-7-O-phosphate only has to be used at 0.1 times the dose of genistein to improve the accumulation of fat. Therefore, genistein-7-O-phosphate is markedly superior to genistein when it comes to weight reduction or inhibition of fat buildup.

### Example 2: Staining of Tissue Sections

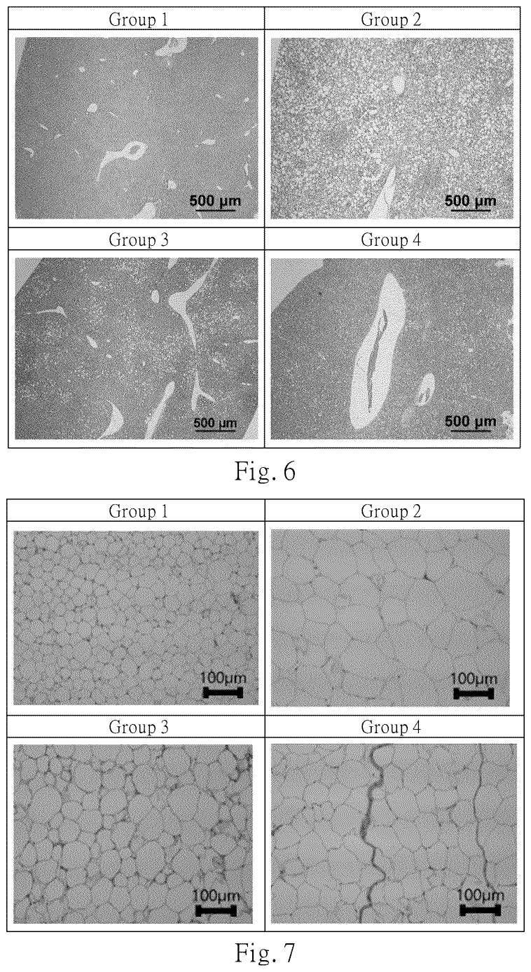

The livers and fat tissues in example 1 were sectioned and stained with hematoxylin and eosin stain (H&E stain), and the results are shown in FIG. 6 and FIG. 7.

It can be known from the sections of group 1 and group 2 in FIG. 6 and FIG. 7 that the high-fat and high-fructose diet caused fat to accumulate in the livers, forming fatty livers, and that the diet also increased the size of the adipocytes in the fat tissues, giving rise to a significant increase in body fat.

It can be further known from the results in FIG. 6 and FIG. 7 that, compared with the mice in group 2, the mice in group 3 and group 4 did not show significant fat buildup in the liver but showed a significant reduction in size of the adipocytes.

It can be inferred from the foregoing results that the administration of genistein or genistein-7-O-phosphate can effectively improve the accumulation of fat in an individual's body and thereby produce the effect of preventing or improving fatty liver, obesity, or a disease related to fat metabolism, and that genistein-7-O-phosphate can be used at a lower dose than genistein to achieve the same effect as genistein. In other words, genistein or genistein-7-O-phosphate can improve or prevent fat buildup or a disease related to fat metabolism.

### Example 3: Biochemical Tests of Blood

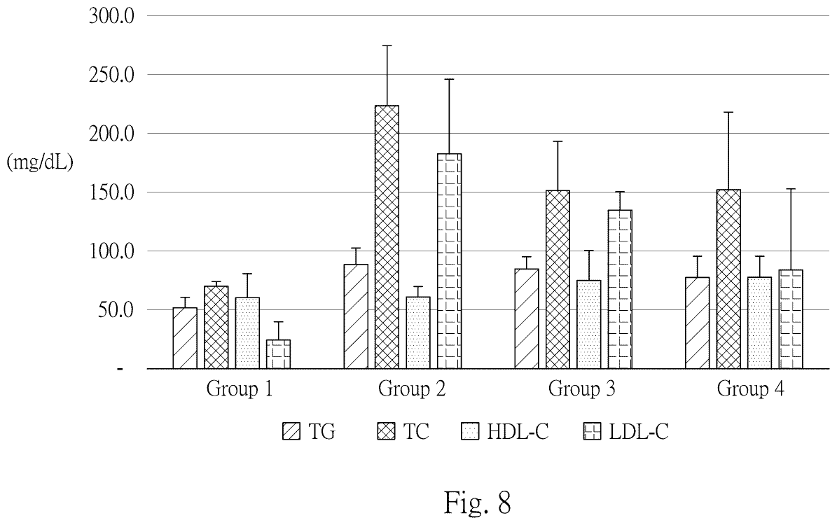

Whole blood was collected from the mice of each group in example 1 and centrifuged at 10,000 g for 10 minutes in order to obtain the serum. A fully automatic serum biochemical analyzer (CoBAS MIRA PLUS, Basel, Switzerland) was then used to measure the serum triglyceride (TG) level, total cholesterol (TC) level, high-density lipoprotein-cholesterol (HDL-C) level, and low-density lipoprotein-cholesterol (LDL-C) level of the serum. The results are shown in FIG. 8.

It can be known from the results in FIG. 8 that, compared with the mice in group 1, the mice in group 2 showed a significant increase in the TC level and LDL-C level of blood, and this indicates that the high-fat and high-fructose diet posed the risk of hyperlipidemia, hypercholesterolemia, or diseases related to either of the above. By contrast, compared with the mice in group 2, the mice in group 3 and group 4 showed a significant reduction in the TC and LDL-C levels of blood.

It can be inferred from the foregoing results that genistein or genistein-7-O-phosphate can inhibit or improve hyperlipidemia and hypercholesterolemia caused by a high-fat and/or high-sugar diet, thereby producing the effect of treating or preventing hyperlipidemia or diseases related thereto and hypercholesterolemia or diseases related thereto, and that due to its higher bioavailability than genistein, genistein-7-O-phosphate can be used at a lower dose than genistein to produce the same effect as genistein.

### Example 4: Hepatic Triglyceride and Cholesterol Tests

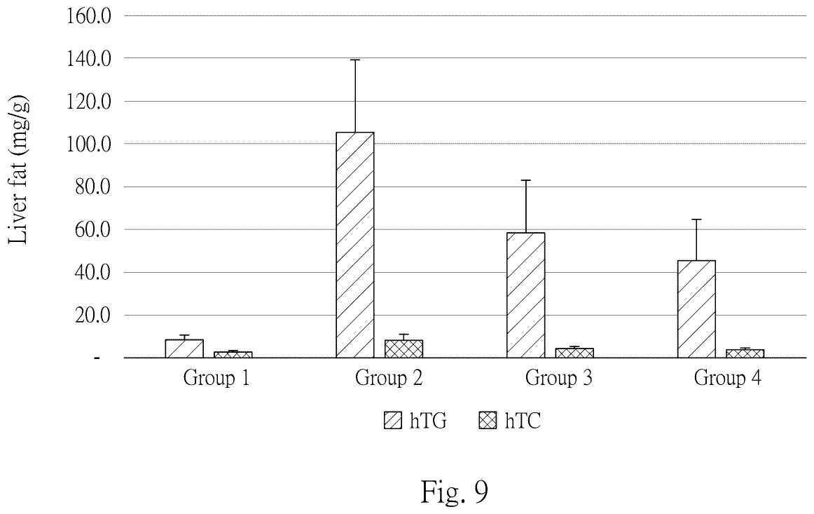

The largest lobe of the liver of each mouse in each group in example 1 was taken and centrifuged at 10,000 g for 10 minutes, and then a fully automatic serum biochemical analyzer (CoBAS MIRA PLUS, Basel, Switzerland) was used to measure the hepatic triglyceride content and hepatic total cholesterol content. The results are shown in FIG. 9.

It can be known from the results in FIG. 9 that, compared with the mice in group 1, the mice in group 2 showed a significant increase in hepatic triglycerides and total cholesterol, and this indicates that the high-fat and high-fructose diet was likely to cause fatty liver or diseases related thereto. By contrast, compared with the mice in group 2, the mice in group 3 and group 4 showed a significant reduction in the hepatic total cholesterol and triglyceride contents.

It can be inferred from the foregoing results that genistein or genistein-7-O-phosphate can inhibit or improve fatty liver (or diseases related thereto) caused by a high-fat and/or high-sugar diet, and that genistein-7-O-phosphate can be used at an extremely low dose to produce the effect of improving or preventing fatty liver or diseases related thereto.

### Example 5: Analysis of Cytokines Related to Liver Inflammation

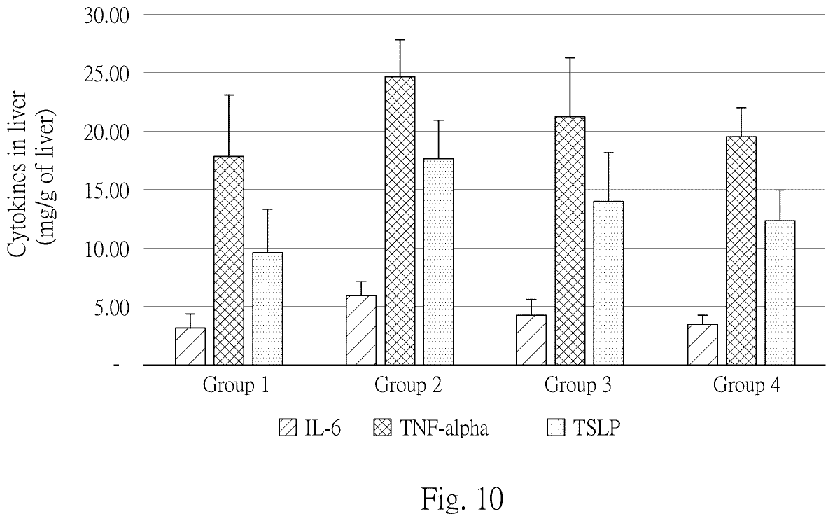

Liver tissue was taken from the mice of each group in example 1, added with an appropriate amount of phosphate-buffered saline buffer solution, homogenized, and then centrifuged at 10,000 g for 10 minutes. After that, the supernatant was collected, and the interleukin 6 (IL-6), thymic stromal lymphopoietin (TSLP), and tumor necrosis factor-a (TNF-α) contents of the liver supernatant were determined with a commercially available enzyme-linked immunosorbent assay (ELISA) kit. The results are shown in FIG. 10.

It can be known from the results in FIG. 10 that, compared with the mice in group 1, the mice in group 2 showed a significant increase in the IL-6, TSLP, and TNF-α contents of their livers, and this indicates that the high-fat and high-sugar diet induced inflammation in the livers of the mice in group 2. By contrast, compared with the mice in group 2, the mice in group 3 and group 4 showed a significant decrease in the inflammation-related hormones in their livers, and this indicates that the continuous provision of the high-fat and high-sugar diet did not cause inflammatory responses in the livers of the mice.

The results in FIG. 10 prove that genistein or genistein-7-O-phosphate can inhibit or improve liver inflammation (or diseases related thereto) caused by a high-fat and/or high-sugar diet, and that the required dose of genistein-7-O-phosphate is 0.1 times that of genistein.

### Example 6: Structural Analysis

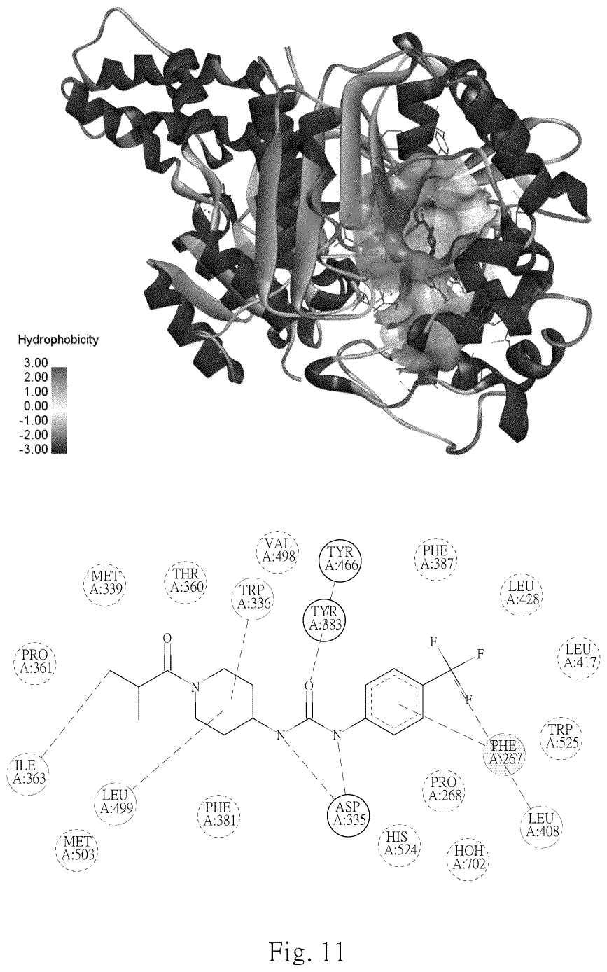

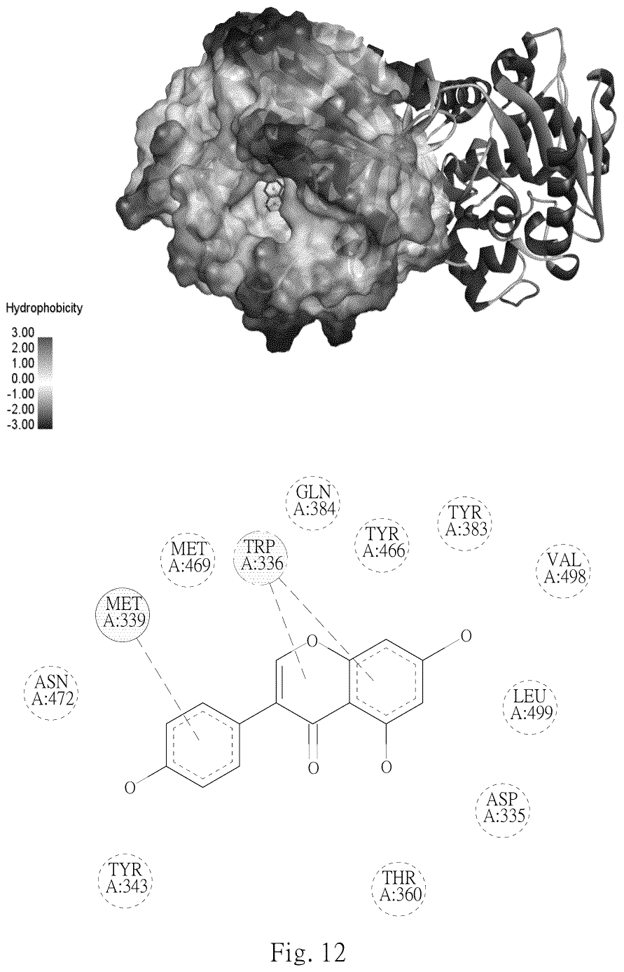

In this example, a protein analysis technique was used to analyze how each of genistein and compound33 docks with an sEH protein molecule, and the results are shown in FIG. 11 and FIG. 12. Also analyzed were the three-dimensional (3D) spatial coordinates and bond energy, and the results are shown in Table 1 below.

TABLE 1
Molecular docking analysis of compound33 and
genistein dock with an sEH protein molecule

| | Compound33 | Genistein |
|---|---|---|
| 3D spatial coordinates | X = 15.357481 | X = 17.745773 |
| | Y = 10.613963 | Y = 7.967000 |
| | Z = 16.643778 | Z = 10.490318 |
| Bond energy (kcal/mol) | −10.3 | −8.7 |

It can be known from the results in Table 1, FIG. 11, and FIG. 12 that genistein as well as the commercially available sEH inhibitor (i.e., compound33) was able to bond to the enzyme sEH, and from this it can be inferred that genistein is indeed capable of inhibiting the activity of sEH. As genistein-7-O-phosphate can be metabolically converted into genistein after entering an individual's small intestine, genistein-7-O-phosphate also has the activity to inhibit the enzyme sEH in an individual's body.

Furthermore, the half-maximal inhibitory concentration (IC₅₀) of genistein against the enzyme sEH was analyzed, and it was found that the IC₅₀ of genistein was 11.45 nM.

What is claimed is:

1. A method of treating and/or preventing a fat-related metabolic disease using genistein (GEN) or genistein-7-O-phosphate (G7P), the method comprising: administering an effective amount of said genistein, said genistein-7-O-phosphate, or a composition containing either of said genistein and said genistein-7-O-phosphate to an individual to inhibit activity of soluble epoxide hydrolase (sEH) in the individual's body, thereby treating and/or preventing the fat-related metabolic disease.

2. The method of treating and/or preventing a fat-related metabolic disease using genistein or genistein-7-O-phosphate as claimed in claim 1, wherein the genistein docks with the soluble epoxide hydrolase after entering the individual's body, thereby inhibiting the activity of the soluble epoxide hydrolase.

3. The method of treating and/or preventing a fat-related metabolic disease using genistein or genistein-7-O-phosphate as claimed in claim 2, wherein the genistein has a half-maximal inhibitory concentration (IC₅₀) of 10-12 nM against the soluble epoxide hydrolase.

4. The method of treating and/or preventing a fat-related metabolic disease using genistein or genistein-7-O-phosphate as claimed in claim 1, wherein the genistein-7-O-phosphate is metabolically converted into the genistein after entering the individual's small intestine so as to inhibit the activity of the soluble epoxide hydrolase.

5. The method of treating and/or preventing a fat-related metabolic disease using genistein or genistein-7-O-phosphate as claimed in claim 1, wherein the fat-related metabolic disease is related to excessive accumulation of fat.

6. The method of treating and/or preventing a fat-related metabolic disease using genistein or genistein-7-O-phosphate as claimed in claim 5, wherein the fat-related metabolic disease is fatty liver, obesity, or hepatitis.

7. The method of treating and/or preventing a fat-related metabolic disease using genistein or genistein-7-O-phosphate as claimed in claim 1, wherein the fat-related metabolic disease is caused by hyperlipidemia.

8. The method of treating and/or preventing a fat-related metabolic disease using genistein or genistein-7-O-phosphate as claimed in claim 1, wherein the composition is a drug, food, or a nutritional supplement.
